(19) United States
(12) Patent Application Publication
(10) Pub. No.: US 2025/0032100 A1
(43) Pub. Date: Jan. 30, 2025

(54) System and Method for Tissue Biopsy Guidance

(71) Applicant: The Board of Regents of the University of Oklahoma, Norman, OK (US)

(72) Inventors: Qinggong Tang, Norman, OK (US); Chongle Pan, Norman, OK (US); Chen Wang, Norman, OK (US); Feng Yan, Norman, OK (US); Paul Calle Contreras, Norman, OK (US); Yunlong Liu, Norman, OK (US)

(21) Appl. No.: 18/912,909

(22) Filed: Oct. 11, 2024

Related U.S. Application Data
(63) Continuation-in-part of application No. 18/428,985, filed on Jan. 31, 2024.
(60) Provisional application No. 63/482,410, filed on Jan. 31, 2023.
(60) Provisional application No. 63/590,310, filed on Oct. 13, 2023.

(51) Int. Cl. A61B 10/02; A61B 17/34; A61B 34/20

(52) U.S. Cl. CPC A61B 10/0275; A61B 17/3403; A61B 2034/2055

(57) ABSTRACT

A method of sampling a target tissue in an animal body, comprising the steps of inserting a endoscope-needle system into the animal body, guiding the endoscope-needle system along a path toward the target tissue using a Doppler-based optical coherence tomography (OCT) system coupled with machine-learning-based computer-aided diagnosis (ML-CAD), wherein the Doppler-based OCT system coupled with ML-CAD enables the identification of blood vessels along the path in advance of the needle tip of the endoscope-needle system thereby substantially avoiding damage to the blood vessels along the path as the needle tip of the endoscope-needle system is guided into the target tissue, after the needle tip is guided into the target tissue, and removing a tissue sample from the target tissue.

### CROSS REFERENCE TO RELATED APPLICATIONS

The present application is a continuation-in-part of U.S. patent application Ser. No. 18/428,985, filed on Jan. 21, 2024, which claims the benefit of U.S. Provisional Patent Application Ser. No. 63/482,410, filed on Jan. 31, 2023. The present application also claims the benefit of U.S. Provisional Patent Application Ser. No. 63/590,310 filed on Oct. 13, 2023. Each of the above applications is hereby expressly incorporated herein by reference in its entirety.

### STATEMENT REGARDING FEDERALLY SPONSORED RESEARCH AND DEVELOPMENT

This invention was made with government support under Grant Numbers OIA-2132161and 2238648 awarded by the National Science Foundation, and Grant Numbers R01DK133717 and P20 GM135009 awarded by the National Institutes of Health. The government has certain rights in the invention.

### BACKGROUND

Tumor biopsy is the most commonly used surgical integral procedure to take tumor tissue. Tumor biopsy followed by histopathology have been widely applied for tumor identification and diagnosis. The biopsied sample will be further utilized to categorize the tumor to be benign, malignant or nondiagnostic. Despite being a common urological procedure, tumor biopsy presents two operational challenges: 1) precisely taking the tumor tissue; and 2) avoiding hemorrhage from blood vessel rupture. Real-time ultrasound guidance technique is currently clinically used in the biopsy needle guidance. It can provide a macroscopic field of view for operators, so it significantly improves the biopsy efficiency. Nevertheless, it is difficult to accurately locate the needle tip and identify the tissue types ahead of biopsy needle, for example in percutaneous renal biopsy (PRB), thus resulting in high false negative rates because of sampling errors. Furthermore, the limited spatial resolution (including Doppler ultrasound) and low contrast for different soft tissues are inadequate for identifying the blood vessels directly in front of the needle tip inside the organs to prevent bleeding complications.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein. The figures are not necessarily to scale and certain features and certain views of the figures may be shown as exaggerated in scale or in schematic in the interest of clarity and conciseness.

FIG. 1 shows a schematic of the forward-viewing OCT probe used to collect data. FC: fiber coupler; PC: polarization controller; MZI: Mach-Zehnder interferometer; BD: balanced detector; DAQ: Data acquisition.

FIG. 2 shows an exemplary computing apparatus for use in the method and system of the present disclosure.

FIG. 3 shows (A) a picture of the human kidney with tumor (inset), and (B) a data acquisition procedure for obtaining tissue sample results using the OCT probe apparatus of FIG. 1.

FIG. 4 shows how OCT imaging can be conducted on a computer monitor for training. Endoscopic images of normal and tumorous tissues are obtained using the OCT probe. OCT imaging results of (A) normal tissues and tumor tissue (scale bar: 250 μm) are compared with (B) corresponding histology images (scale bar: 500 μm).

FIG. 5 shows attenuation coefficient distribution results between normal tissue and tumor tissue (A), and ROC curves of the classification with attenuation coefficient between tumor tissue and other tissues (B).

FIG. 6 shows attenuation coefficient distribution results of all five tissue types (A), and ROC curves of the classification with attenuation coefficient among different normal tissues (B).

FIG. 7 shows a schematic of a polarization-sensitive OCT (PS-OCT) system as used in Example 2. GSM represents a Galvanometer scanning mirror.

FIG. 8 is an alternate schematic of the PS-OCT endoscopic probe of FIG. 7. GSM: Galvanometer scanning mirror

FIG. 9 shows results of PS-OCT imaging of fat, muscle, and nerve sarcoma tissues and normal tissue.

FIG. 10 is a schematic representation of a CNN system used to automate the classification of normal tissue and sarcoma tissue recognition procedure to obtain the results in FIG. 9.

FIG. 11 shows PS-OCT imaging results of normal liver tissue and cancerous liver tissue.

FIG. 12 shows distribution results of the attenuation coefficient method for distinguishing liver tumor and normal tissue (A), and a Confusion matrix showing the quantitative results (B).

### DETAILED DESCRIPTION

The present disclosure is directed, in at least one embodiment, to a novel needle-based guidance system for tissue biopsy procedures. In certain embodiments the tissue sample is obtained from a cancerous tumor. The biopsy guidance system uses structural Optical Coherence Tomography (OCT) to address and mitigate the sampling error of biopsy by recognizing and differentiating normal tissues and tumor tissue. Doppler OCT is used to address and mitigate bleeding complications by visualizing the blood vessels in front of the needle in real time. By using forward-imaging needle OCT with Doppler capability, the system uses a miniaturized OCT probe together with machine-learning-based computer-aided diagnosis (ML-CAD) software.

This innovative imaging platform automatically detects and recognizes the tissue structures (e.g., renal and liver tissue structures) and the at-risk blood vessels ahead of the biopsy needle (i.e., before the needle tip reaches the at-risk blood vessels and tissues). The disclosed system and method comprise an OCT/CAD platform which improves the biopsy accuracy and reduces the complication rates of needle placement to less than 5% and substantially eliminates accidental blood vessel ruptures during the biopsy procedure (e.g., tumor biopsy).

In certain embodiments, the target tissue sample that is obtained during the biopsy procedure is taken from a breast, kidney, liver, bone, bone marrow, colon, small intestine, stomach, esophagus, spleen, lung, pancreas, rectum, adrenal gland, thyroid, parathyroid, pituitary, heart, muscle, peritoneum, endometrium, eye, bladder, penis, prostate, testicle, uterus, ovary, cervix, vulva, fallopian tube, skin, urethra, spine, brain, head, neck, throat, tongue, larynx, pharynx, lymph glands, thymus, nerve, fatty tissue, and/or central nervous system. In certain embodiments, the target tissue that is obtained during the biopsy procedure is from a tumor, such as a benign tumor or cancerous tumor. The tumor may be located in one or more of the breast, kidney, liver, bone, bone marrow, colon, small intestine, stomach, esophagus, spleen, lung, pancreas, rectum, adrenal gland, thyroid, parathyroid, pituitary, heart, muscle, peritoneum, endometrium, eye, bladder, penis, prostate, testicle, uterus, ovary, cervix, vulva, fallopian tube, skin, urethra, spine, brain, head, neck, throat, tongue, larynx, pharynx, lymph glands, thymus, nerves, fatty tissues, and central nervous system.

Before further describing various embodiments of the apparatus, component parts, and methods of the present disclosure in more detail by way of exemplary description, examples, and results, it is to be understood that the embodiments of the present disclosure are not limited in application to the details of apparatus, component parts, and methods as set forth in the following description. The embodiments of the apparatus, component parts, and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the apparatus, component parts, and methods of the present disclosure have been described in terms of particular embodiments, it will be apparent to those of skill in the art that variations may be applied to the apparatus, component parts, and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, including but not limited to U.S. patent application Ser. No. 17/530,131, filed Nov. 18, 2021, U.S. patent application Ser. No. 18/428,985, filed on Jan. 21, 2024, U.S. Provisional Patent Application Ser. No. 63/482,410, filed Jan. 31, 2023, and U.S. Provisional Patent Application Ser. No. 63/115,452, filed Nov. 18, 2020, each of which is hereby expressly incorporated by reference herein in its entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

The following abbreviations may be used herein:
ASIC: application-specific integrated circuit
AUC: area under the ROC curve
BD: balanced detector
BE: Barrett's esophagus
CAD: computer-aided diagnosis
CCD: charge-coupled device
CNN: convolutional neural network
CPU: central processing unit
CT: computed tomography
DAQ: data acquisition
dB: decibel(s)
DOCT: doppler optical coherence tomography
DOPU: degree of polarization uniformity
DSP: digital signal processor
EO: electrical-to-optical
FC: fiber coupler
FOV: field of view
FPGA: field-programmable gate array
G: gauge
GI: gastrointestinal
GRAD-CAM: gradient-weighted class activation mapping
GRIN: gradient-index
GSM: galvanometer scanning mirror
H&E: hematoxylin and eosin
HMP: hypothermic machine perfusion
kHz: kilohertz
LOR: loss of resistance
MEMS: microelectromechanical systems
mIoU: mean intersection-over-union
ML: machine learning
ML-CAD: machine learning computer-aided diagnosis
mm: millimeter(s)
MRI: magnetic resonance imaging
ms: millisecond(s)
mW: milliwatt(s)

MZI: Mach-Zehnder interferometer
nm: nanometer(s)
OCT: optical coherence tomography
OE: optical-to-electrical
PC: polarization controller
PCN: percutaneous nephrostomy
PCNL: percutaneous nephrolithotomy
PET: Positron emission tomography
PS-OCT: polarization-sensitive optical coherence tomography
PT: pre-trained
PRB: percutaneous renal biopsy
RAM: random-access memory
RCC: renal cell carcinoma
ResNet: residual neural network
RF: radio frequency
RI: randomly-initialized
ROM: read-only memory
ROC: receiver operating characteristic
RX: receiver unit
SGD: stochastic gradient descent
SRAM: static RAM
SS-OCT: swept-source OCT
TCAM: ternary content-addressable memory
TX: transmitter unit
2D: two-dimensional
3D: three-dimensional
μm: micrometer(s)
°: degree(s).

As utilized in accordance with the methods and compositions of the present disclosure, the following terms and phrases, unless otherwise indicated, shall be understood to have the following meanings: The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8,9, 10, 15, 20, 30, 40, 50, 100, or any integer inclusive therein. The phrase “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the apparatus, composition, or the methods or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately”, where used herein when referring to a measurable value such as an amount, percentage, temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 75% of the time, or at least 80% of the time, or at least 85% of the time, or at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 75%, or at least 80%, or at least 85%, or at least 90%, or at least 91%, or at least 92%, or at least 93%, or at least 94%, or at least 95%, or at least 96%, or at least 97%, or at least 98%, or at least 99%, of the referenced dimension or measurement (e.g., length).

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20,etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60,60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100units to 1000 units, 100 units to 500 units, 200 units to 1000 units, 300 units to 1500 units, 400units to 2000 units, 500 units to 2000 units, 500 units to 1000 units, 250 units to 1750 units, 250units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure. More particularly, a range of 10-12 units includes, for example, 10, 10.1, 10.2, 10.3, 10.4, 10.5, 10.6, 10.7, 10.8, 10.9, 11.0, 11.1, 11.2, 11.3, 11.4, 11.5,11.6, 11.7, 11.8, 11.9, and 12.0, and all values or ranges of values of the units, and fractions of the values of the units and integers within said range, and ranges which combine the values of the boundaries of different ranges within the series, e.g., 10.1 to 11.5.

Methods and apparatus disclosed and described in U.S. Patent Application Ser. No. 17/530, 131, filed Nov. 18, 2021, U.S. patent application Ser. No. 18/428,985, filed on Jan. 21, 2024, U.S. Provisional Patent Application Ser. No. 63/482,410, filed Jan. 31, 2023, and U.S. Provisional Patent Application Ser. No. 63/115,452, filed Nov. 18, 2020, may be used in the methods and systems of the present disclosure.

As used herein any reference to “we” as a pronoun may include laboratory personnel or other contributors who assisted in the laboratory procedures and data collection and is not intended to represent an inventorship role by said laboratory personnel or other contributors in any subject matter disclosed herein.

While several embodiments have been provided in the present disclosure, it may be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, components, techniques, or methods without departing from the scope of the present disclosure. Other items shown or discussed as coupled may be directly coupled or may be indirectly coupled or communicating through some interface, device, or intermediate component whether electrically, mechanically, or otherwise. Other examples of changes, substitutions, and alterations are ascertainable by one skilled in the art and may be made without departing from the spirit and scope disclosed herein.

### EXAMPLE 1

### Materials and Methods

### Experimental Setup

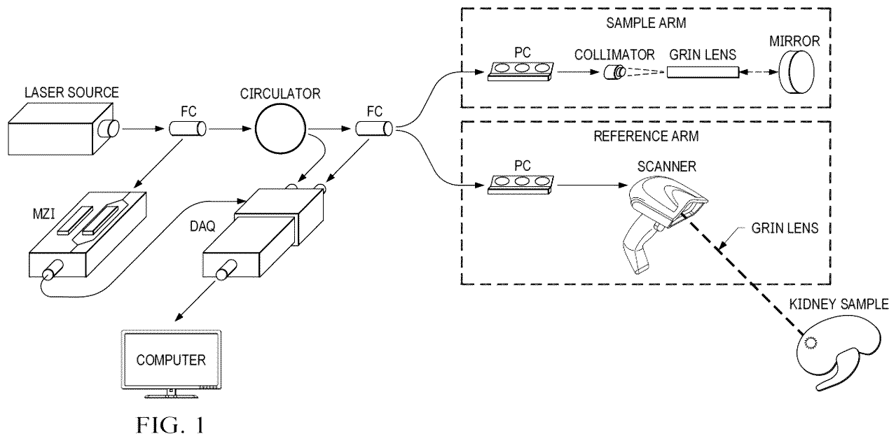

A forward-viewing OCT probe based on a swept-source OCT (SS-OCT) system. The light source utilized was a 1300 nm swept-source laser with a bandwidth of 100 nm. The scanning rate of our system reaches 200 kHz. As illustrated in FIG. 1, the light was initially divided using a fiber coupler (FC) into two distinct paths: one directed with 97% of power towards a circulator for the OCT imaging, and the other with 3% power directed to an MZI to initiate the sampling process. Upon traversing the circulator, the laser underwent another division through another FC into a reference arm and a sample arm. Subsequently, the light reflected by the mirror in the reference arm and the light backscattered by the tissue in the sample arm converged, generating an interference signal. This signal was then denoised using a balanced detector (BD), then acquired by the data acquisition (DAQ) board and processed and visualized on a computer in the end. Polarization controllers (PCs) were employed in both arms to mitigate background noise.

In PRB procedures, 14 G or 16 G biopsy needles are commonly used. To assess feasibility, in a non-limiting embodiment we employed GRIN lenses with a diameter of 1.3 mm and length of 138 mm, allowing them to be accommodated within the 14 G or 16 G biopsy needles. These GRIN lenses possess an RI distribution that varies perpendicular to the optical axis, facilitating effective image transmission from one end to the other.

In the presently disclosed, non-limiting configuration, we positioned a GRIN lens in the sample arm ahead of the OCT scanning lens for endoscopic imaging. Additionally, an identical GRIN lens was situated in the reference arm to counteract dispersion. The proximal end of the GRIN lens in the sample arm was positioned at the focal point of the scanner lens, maximizing the delivery of light to the sample. In a non-limiting embodiment, the system features an axial resolution of approximately 11 μm and a lateral resolution of around 20 μm. It provides a sensitivity of 92 dB and covers an FOV of approximately 1.25 mm in diameter.

### Exemplary Computing Apparatus

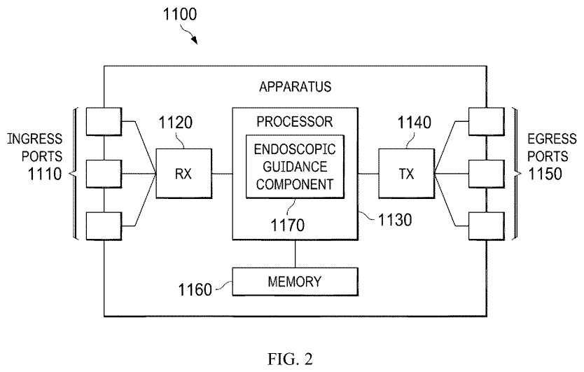

FIG. 2 shows a schematic diagram of an apparatus 1100 according to one non-limiting embodiment of the present disclosure. The apparatus 1100 may implement the disclosed embodiments of the OCT system of the present disclosure. The apparatus 1100 comprises ingress ports 1110 and an RX 1120 to receive data; a processor 1130, or logic unit, baseband unit, or CPU, to process the data; a TX 1140 and egress ports 1150 to transmit the data; and a memory 1160 to store the data. The apparatus 1100 may also comprise OE components, EO components, or RF components coupled to the ingress ports 1110, the RX 1120, the TX 1140, and the egress ports 1150 to provide ingress or egress of optical signals, electrical signals, or RF signals.

The processor 1130 is any combination of hardware, middleware, firmware, or software. The processor 1130 comprises any combination of one or more CPU chips, cores, FPGAs, ASICs, or DSPs. The processor 1130 communicates with the ingress ports 1110, the RX 1120, the TX 1140, the egress ports 1150, and the memory 1160. The processor 1130 comprises an endoscopic guidance component 1170, which implements the disclosed embodiments. The inclusion of the endoscopic guidance component 1170 therefore provides a substantial improvement to the functionality of the apparatus 1100 and effects a transformation of the apparatus 1100 to a different state. Alternatively, the memory 1160 stores the endoscopic guidance component 1170 as instructions, and the processor 1130 executes those instructions.

The memory 1160 comprises any combination of disks, tape drives, or solid-state drives. The apparatus 1100 may use the memory 1160 as an over-flow data storage device to store programs when the apparatus 1100 selects those programs for execution and to store instructions and data that the apparatus 1100 reads during execution of those programs. The memory 1160 may be volatile or non-volatile and may be any combination of ROM, RAM, TCAM, or SRAM.

A computer program product may comprise computer-executable instructions for storage on a non-transitory medium and that, when executed by a processor, cause an apparatus to perform any of the embodiments. The non-transitory medium may be the memory 1160, the processor may be the processor 1130, and the apparatus may be the apparatus 1100.

### Data Acquisition

The experimental work was approved by the University of Oklahoma Institutional Review Board. Written informed consent was obtained by Lifeshare of Oklahoma. Patient demographics were collected upon obtaining consent. For our dataset, we utilized five human kidney samples and five renal carcinoma samples. OCT images of carcinoma tissue and different normal tissues were imaged by our OCT probe. To keep the organ as fresh as possible, the kidney was preserved by HMP, and we started the experiment as soon as the sample was acquired.

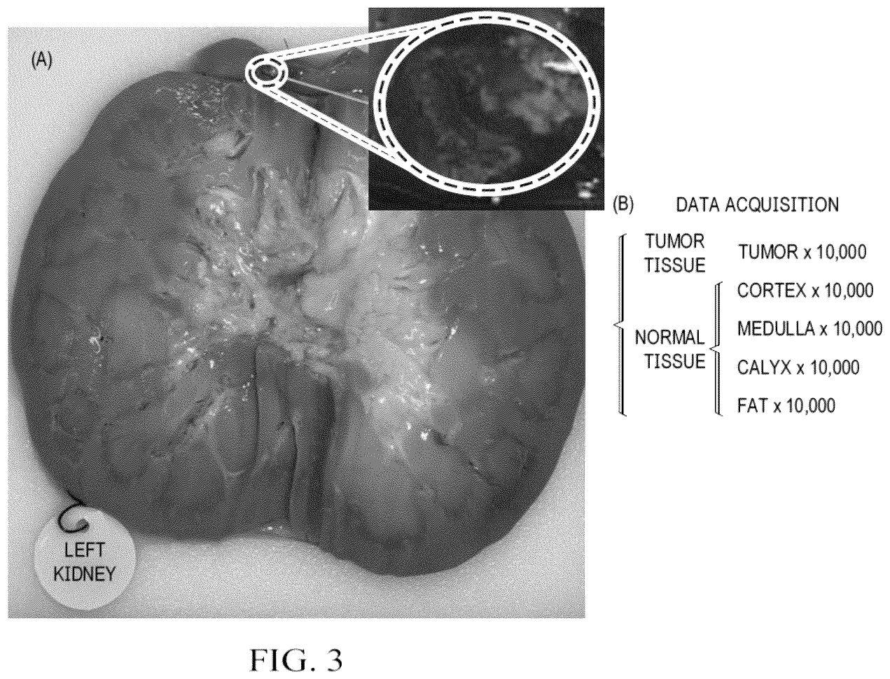

FIG. 3(A) illustrates a kidney sample with carcinoma, clearly showcasing the tumor tissue alongside other normal renal tissues including cortex, medulla, calyx, fat, and pelvis. To emulate a practical biopsy procedure during the experiment, we carefully inserted the OCT probe into different renal tissue types, applying controlled force to compress the tissue. OCT image acquisition was successfully performed.

For quantitative identification of tumor tissue, we utilized both conventional methods, involving the calculation of attenuation coefficients, and deep learning techniques for automated recognition of renal tissue types. To train the machine learning model, we obtained a dataset of 10,000 OCT images from the tumor and five different normal renal tissues, respectively (FIG. 3(B)). The size of all output images was set as lateral X depth: 650×1050 (X×Z: 1.30 mm×2.10 mm), so each result covered the same FOV for attenuation coefficient method and CNN process.

### Attenuation Coefficient Method for Tissue Recognition

The results provide in-depth structural insights into renal tissues. The attenuation coefficient serves as parameter which signifies the decrease in signal intensity observed in OCT images as depth increases. In structural OCT images, the imaging intensity reflects the backscattering level of the tissue sample, so different attenuation coefficients demonstrate different tissue features and help the tissue classification. The use of attenuation coefficients for cancer detection has been documented. Compared to normal tissue, cancerous tissue is typically denser, thus its imaging signal value attenuates faster in a vertical direction as represented by a higher attenuation coefficient. The attenuation coefficient of each A-scan can be extracted and used to generate a spatial attenuation coefficient distribution, which helps visually distinguish tumor and normal tissues.

Here we used Beer-Lambert law to describe the structural OCT intensity signals in depth as follows:

$${I}_{( z )} = {I}_{0} {e}^{- 2 μ z} \qquad ( 1 )$$

where I₀ represents the initial incident light intensity, μ is the attenuation coefficient value which represents the decay rate, and z is the depth. Thus, μ can be described as:

$$μ = - \frac{1}{2} \cdot \frac{d log {I}_{( Z / 0 )}}{{d}_{z}} \qquad ( 2 )$$

I_{(Z/0)} is the ratio of recorded OCT intensity in depth to the initial incident light intensity. The intrinsic optical attenuation coefficient was calculated according to the slope of OCT intensity in a 250-pixel depth window.

CNN Methods for Tissue Identification

In this example, we evaluated two CNN architectures: ResNet50 and InceptionV3.ResNet50 has about 25.6 million parameters and InceptionV3 has about 22 million parameters. During the data processing, the data was divided into three separate datasets: three subjects as training dataset, one as validation dataset and another one subject as testing dataset. This division enabled us to obtain a more accurate estimation of the generalization error, as the testing dataset remained independent of both training and parameter selection processes. Subsequently, we executed a 5-fold cross-validation procedure. Early stopping was implemented with a patience of 20. The chosen optimizer was Stochastic Gradient Descent with Nesterov accelerated gradient, utilizing a momentum value of 0.9 and a learning rate of 0.01. Additionally, the learning decay rate was set to be 0.1. The average epoch count from the cross-validation folds was subsequently employed to train the model intended for use on the test dataset. To evaluate the performance of our models, we implemented a 5-fold cross-testing, where each kidney was sequentially designated as the testing dataset while the remaining four kidneys constituted the training set. This approach ensured that every kidney in our dataset was utilized for testing purposes, thereby enhancing the model's capacity for generalization.

Three metrics including precision, recall, and F₁ score were utilized to benchmark the classification performance. Formula of these parameters are listed below:

$$Precision = TP / ( TP + FP ) \qquad ( 3 )$$

$$Recall = TP / ( TP + FN ) \qquad ( 4 )$$

$${F}_{1} score = 2 \cdot \frac{Precision \cdot Recall}{Precision + Recall} = \frac{TP}{TP + \frac{1}{2} ( FP + FN )} \qquad ( 5 )$$

where TP is true positive, FP is false positive, and FN is false negative.

### Results

### Imaging Results of Renal Tumor and Normal Tissues

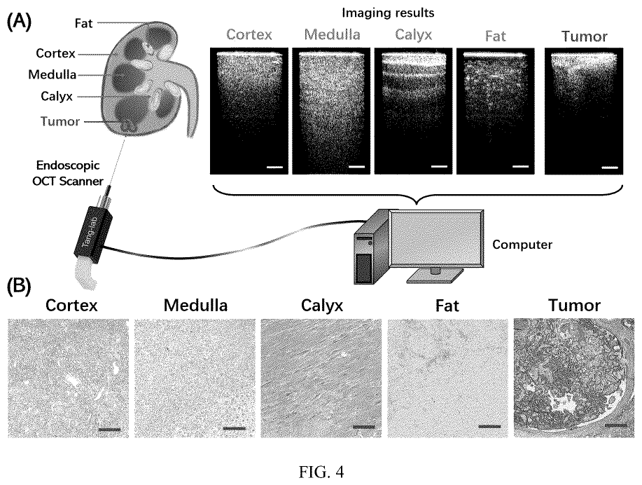

FIG. 4 illustrates the OCT imaging results obtained through our OCT probe, showcasing both renal carcinoma tumor and five distinct normal renal tissues. Two dimensional (2D) cross-sectional OCT images of these tissues demonstrate different structural features (FIG. 4(A)).

In the OCT results, there is a bright line on the top which corresponds to the surface of the GRIN lens. The intensity signal distributions of both cortex and medulla are relatively homogeneous, although the imaging depth of medulla surpasses that of cortex. Calyx exhibits alternately shaded stripes, a result of its transitional epithelium and fibrous tissue composition. Fat presents the darkest pattern, accompanied by bright dots characteristic of adipocytes. Pelvis, as an empty space for collecting urine, has no tissue signal under the GRIN lens surface. The tumor, being the pivotal tissue type for identification during PRB, showcases the shallowest imaging depth while presenting the highest brightness among all OCT results. Its structural features exhibit irregularities. The corresponding histology results were also presented (FIG. 4(B)). These tissues showed different tissue structures and distributions and correlated well with their OCT results. Different normal renal tissues and tumor tissues can be categorized based on their distinctive OCT imaging features. These results demonstrate the ability of the present endoscopic OCT probe to assist in the recognition of tissue types ahead of the needle during PRB procedures.

### Classification Using Attenuation Coefficient Method

First we assessed the performance of attenuation coefficient method indistinguishing renal tumor and normal tissues. We applied tumor tissue and other four normal tissue types in the attenuation coefficient method. A total of 50,000 OCT images (after removing pelvis category), were processed with each tissue type contributing 10,000 images. The performance of the results was demonstrated using the cross-testing approach. Four kidneys were randomly selected as the training dataset, while the remaining kidney served as the testing dataset. To calculate the attenuation coefficient, the region of interest (ROI) window was selected to be 200×250 pixels for each OCT image. The ROIs were chosen from the central sections of the images and the coordinate of the area is selected in the top middle position which includes most tissue information. The mean value of all the attenuation coefficient results from every vertical direction were used to signify the attenuation coefficient level for each OCT image.

Table 1 displays the attenuation coefficient distributions for the five tissue types (four normal renal tissues and tumor tissue). Firstly, considering cortex, medulla, calyx, and fat are all normal tissues, these four types were initially consolidated into a single classification. In this testing, all the tumor tissue images from 5 samples (50,000 images in total) were utilized. To make the number of normal tissues same to tumor, we randomly selected 2,500 images from each normal tissue in every sample (2,500 images×4 tissue types×5 samples=50,000 images).

TABLE 1
Confusion matrix (Attenuation coefficient distributions)
for four normal renal tissues and tumor tissue.

| Tissue Type | Cortex | Medulla | Calyx | Fat | Tumor |
|---|---|---|---|---|---|
| Cortex | 9145 | 14 | 723 | 0 | 118 |
| Medulla | 3 | 9154 | 756 | 87 | 0 |
| Calyx | 4326 | 2820 | 1947 | 347 | 560 |
| Fat | 1895 | 1872 | 1083 | 4942 | 208 |
| Tumor | 17 | 0 | 0 | 0 | 9983 |

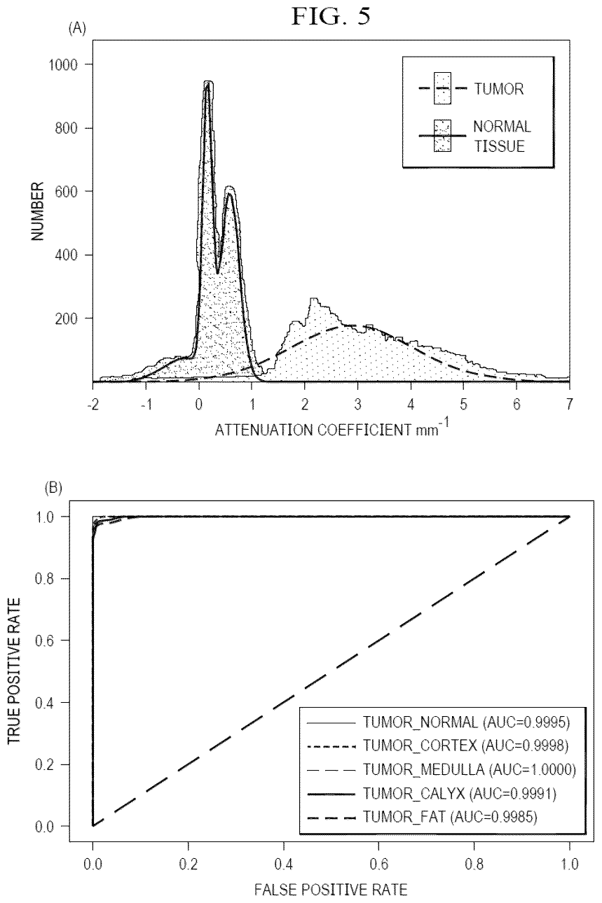

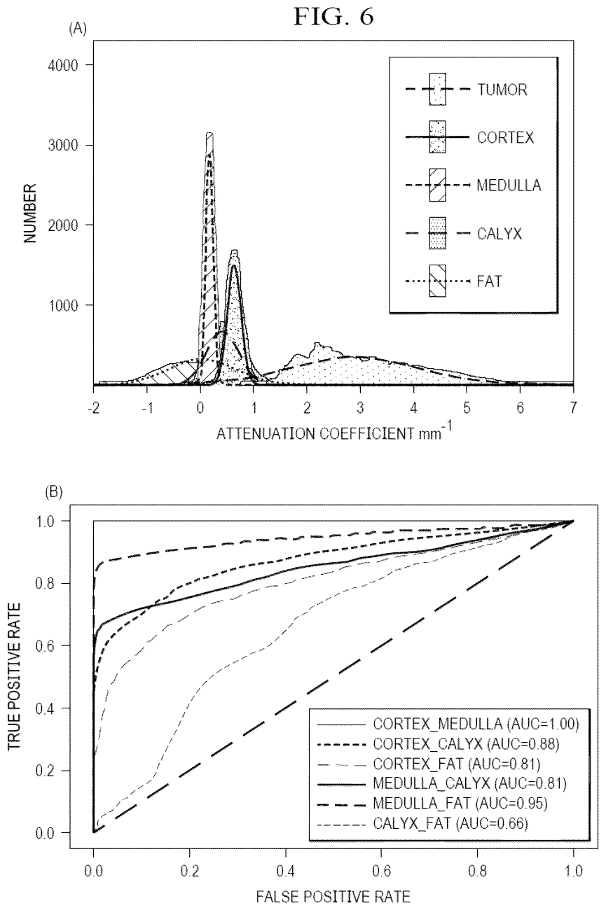

In general, the tumor tissue and the normal tissue types displayed distinctive distribution characteristics, thereby enabling a clear differentiation between tumor and normal tissues as shown in FIG. 4(A-B). To analyze these distributions, curve fitting was employed, and the resulting fitted normal distributions for the attenuation coefficient were visually depicted as shown in FIG. 5(A). To quantify the separation of the tumor from normal tissues, the cross point of two curves was chosen as threshold and used to evaluate the accuracy. From the receiver operating characteristic (ROC) curves results between tumor and normal renal tissues in FIG. 5(B), the area under the curves (AUC) values were over 99.95%, which proved the tumor recognition capacity of using attenuation coefficients. After that, we evaluated each renal tissue type by using 10,000 OCT images from 5 samples (50,000 images in total). All data were utilized, and the results were shown in FIG. 6(A).

From the ROC curves results between tumor and each normal renal tissues in FIG. 6(B), the AUC values were all over 99.8%, which further prove the tumor recognition capacity of using attenuation coefficients. The different prediction parameters also showed very promising results of tumor identification, providing 98.19% of accuracy, 90.84% of precision, 99.83% of recall, and 95.10% of F1 score. However, different normal renal tissues are difficult to classify. The confusion matrix is shown in Table 1, and the corresponding prediction results are demonstrated in Table 2.

TABLE 2
Prediction results corresponding to
Confusion matrix scores of Table 1.

| Tissue | Accuracy | Precision | Recall | F₁ Score |
|---|---|---|---|---|
| Type | (%) | (%) | (%) | (%) |
| Cortex | 84.26 | 57.78 | 91.49 | 70.63 |
| Medulla | 88.16 | 70.01 | 91.55 | 79.13 |
| Calyx | 79.51 | 48.14 | 19.47 | 27.16 |
| Fat | 90.57 | 89.58 | 49.42 | 60.47 |
| Tumor | 98.19 | 90.84 | 99.83 | 95.10 |

Different prediction parameters showed very low rates in normal tissue recognition, especially for calyx. Moreover, from the ROC curves shown in FIG. 6(B), the AUCs were 100% for cortex—medulla, 88% for cortex—calyx, 81% for cortex—fat, 86% for medulla—calyx, 95% for medulla—fat, and 66% for calyx—fat. The needle's precise placement can furnish medical professionals with additional procedural insights, enhancing surgical efficiency and overall effectiveness. The present results primarily contribute to tumor recognition, yet the performance in identifying other normal kidney tissues still needed improvement. Consequently, to attain improved classification and accurate identification of various normal tissue types for enhanced needle localization, addition methods were employed.

Tissue Recognition Results Using CNN

The recognition accuracy of kidney tissues was further evaluated through the utilization of the ResNet50 and InceptionV3 CNN models. All comprehensive results, including confusion matrixes and prediction evaluations are presented in Tables 3 and 4 for the ResNet50 model and Tables 5 and 6 for the Inception V3 model.

TABLE 3
Confusion matrix (Attenuation coefficient
distribution) results using ResNet50 model.

| Tissue Type | Cortex | Medulla | Calyx | Fat | Pelvis | Tumor |
|---|---|---|---|---|---|---|
| Cortex | 9875 | 75 | 23 | 27 | 0 | 0 |
| Medulla | 43 | 9956 | 1 | 0 | 0 | 0 |
| Calyx | 136 | 15 | 9553 | 148 | 0 | 148 |
| Fat | 199 | 0 | 56 | 9745 | 0 | 0 |
| Pelvis | 0 | 0 | 21 | 0 | 9969 | 10 |
| Tumor | 123 | 0 | 0 | 9 | 2 | 9866 |

TABLE 4
Prediction evaluation results corresponding
to Confusion matrix scores of Table 3.

| Tissue | Accuracy | Precision | Recall | F₁ Score |
|---|---|---|---|---|
| Type | (%) | (%) | (%) | (%) |
| Cortex | 98.96 | 95.16 | 98.75 | 96.92 |
| Medulla | 99.78 | 99.10 | 99.56 | 99.33 |
| Calyx | 99.09 | 98.95 | 95.53 | 97.21 |
| Fat | 99.27 | 98.15 | 97.45 | 97.80 |
| Pelvis | 99.95 | 99.98 | 99.69 | 99.83 |
| Tumor | 99.51 | 98.43 | 98.66 | 98.54 |

TABLE 5
Confusion matrix (Attenuation coefficient distribution)
results using InceptionV3 model.

| Tissue Type | Cortex | Medulla | Calyx | Fat | Pelvis | Tumor |
|---|---|---|---|---|---|---|
| Cortex | 9964 | 1 | 1 | 34 | 0 | 0 |
| Medulla | 15 | 9978 | 7 | 0 | 0 | 0 |
| Calyx | 69 | 38 | 9639 | 0 | 0 | 254 |
| Fat | 121 | 0 | 30 | 9822 | 0 | 27 |
| Pelvis | 0 | 11 | 15 | 1 | 9973 | 0 |
| Tumor | 10 | 0 | 22 | 0 | 0 | 9968 |

TABLE 6
Prediction evaluation results corresponding
to Confusion matrix scores of Table 5.

| Tissue | Accuracy | Precision | Recall | F₁ Score |
|---|---|---|---|---|
| Type | (%) | (%) | (%) | (%) |
| Cortex | 99.58 | 97.89 | 99.64 | 98.76 |
| Medulla | 99.88 | 99.50 | 99.78 | 99.64 |
| Calyx | 99.27 | 99.22 | 94.39 | 96.74 |
| Fat | 99.65 | 99.64 | 98.22 | 98.92 |
| Pelvis | 99.95 | 100.00 | 99.73 | 99.86 |
| Tumor | 99.48 | 97.26 | 99.68 | 98.46 |

In the case of ResNet50, the tumor recognition accuracy reached 99.51%. Upon analyzing the confusion matrix, we found that most misclassifications of tumor samples were erroneously categorized as cortex. For other normal tissue classification, the accuracy was notably high, with cortex prediction accuracy at 98.96%, medulla at 99.78%, calyx at 99.09%, fat at 99.27%, and pelvis at 99.95%. It should be noticed that none of cortex, medulla and fat images were predicted to be tumor, and calyx was the relatively most likely to be predicted as tumor by mistake.

Utilizing the InceptionV3 model, tumor prediction accuracy reached 99.48%, with remarkable accuracy for normal tissue classification: cortex at 99.58%, medulla at 99.88%, calyx at 99.27%, fat at 99.65%, and pelvis at 99.95%. Calyx recognition has the lowest accuracy and the most misclassifications of tumor samples were erroneously categorized as calyx, which is different from the ResNet model. In terms of the ROC analysis in ResNet50 and InceptionV3, all curves displayed overlapping features (not shown), signifying remarkable AUC values of over 99.9% for all tissues.

The remarkable performance of both models could be attributed to inherent dataset homogeneity. Inception V3 outperformed ResNet50 in overall precision for the all the renal tissues, except with a slightly lower precision in tumor classification. For the recall evaluation, all normal tissues and tumor results using Inception V3 have higher recall values than ResNet50 except calyx. F1 score evaluations in both models show similar trends to recall, while the tumor in Inception V3is slightly lower than ResNet50.

Additionally, the average heatmaps of all the images in the two models were obtained (not shown). Heatmaps can help visualize the regions the models utilized in distinguishing different tissue types. The importance area of the tumor was focused on the very narrow area below the GRIN lens surface line. For the pelvis, the focused area was separated into two parts: a narrow part on the top and a wide area on the bottom. Heatmaps of the other four tissues all focused on the top half areas, constituent with the corresponding signal distribution patterns. The important area of cortex was smaller than that of medulla, calyx, and fat. The brightness of fat is the lowest. Medulla and calyx showed similar features, while there were slight horizontal patterns on the bottom. For model comparison, the highlighted importance areas of InceptionV3 were sparser and wilder, while the areas of ResNet50 were sharper. This was because that the feature map of InceptionV3 tends to capture features at different scales and levels of abstraction and it may produce more diverse and multi-scale features. On the contrary, ResNet50's feature map is more focused and localized within each residual block, so it can capture fine-grained details in the images.

### Discussion

Although PRB is the most commonly performed procedure for evaluating renal tissue, it has remained a challenge even for experienced urologists to precisely extract tumor tissue with minimal sampling error. In the present example, the use of a forward-viewing OCT probe for RCC detection on ex-vivo human kidney was tested for the first time. To validate the system, human kidneys with renal carcinoma were utilized in our experiments. The renal tumor tissue and different normal renal tissues, including cortex, medulla, calyx, and renal fat, were all utilized for testing the guiding function of our system. We applied the probe on these tissues and obtained their OCT images separately. The OCT results of different renal tissues could be classified based on their distinct imaging characteristics. Moreover, the designed OCT probe's dimensions were set to match the PRB needle's size (hollow bore), enabling seamless integration without introducing any additional invasiveness. During the clinical situation, the OCT probe can perform real-time tissue imaging in front of the PRB needle, effectively identifying the tissue type at the needle's tip. To automate the process of tissue identification, we utilized and compared two classification methods in our work: attenuation coefficient and deep learning.

The attenuation coefficient method has been widely used in OCT based cancer diagnosis, including breast cancer, prostate cancer, bladder cancer, colon cancer, etc. Renal mass diagnosis using OCT was also reported, and it exhibited highly promising outcomes in predicting RCC. For the classification task, we imaged 10,000 2D OCT images from renal tumor, cortex, medulla, calyx, and renal fat, respectively. The work leveraged the attenuation coefficient for both tumor recognition and the identification of normal tissues. Recognition of tumor against other normal tissues was detected with the accuracy of 98.19%. While the results were generally favorable, errors of misrecognition still happened. Additionally, relying solely on the attenuation coefficient proved challenging when it came to distinguishing between different types of normal renal tissues. The classification accuracy results were not satisfactory, particularly for the calyx prediction accuracy which fell below 80%.

Accurate identification of normal renal tissues prior to biopsy needle insertion played a pivotal role in precisely tracking the needle's location. To address this important need, we leveraged the capabilities of deep learning methods. Specifically, we employed two well-established CNN architectures, ResNet50 and InceptionV3, both of which demonstrated robust performance in tumor recognition and the classification of various normal renal tissues.

Remarkably, ResNet50 and InceptionV3 achieved tumor recognition rates of 99.51% and 99.48%, respectively, both of which surpassed the performance of the attenuation coefficient method. Equally impressive was the accuracy in normal tissue recognition, with rates consistently exceeding 99% across all tissue types in both models (except cortex prediction using ResNet50 of 98.96%). Our analysis of ROC curves for both models revealed overlapping curves and AUC values consistently exceeding 99.9%. This observation underscores the effectiveness of the present CNN-based approach in the precise classification of different normal renal tissues compared to the attenuation coefficient method. The outstanding performance of these models can be attributed, in part, to their substantial parameter sets. ResNet50 and InceptionV3 feature 25.6 million and 22 million parameters, respectively, in contrast to the single parameter employed by the attenuation coefficient method.

All computations were conducted on a server equipped with two NVIDIA RTX A6000graphics cards, each boasting 48 GB of GDDR6 memory. In terms of training, the cross-validation required an average of 1435 minutes per fold, while the testing phase demanded 1424 minutes. During inference stage with 320 images, ResNet50 reached an average processing time of 18 ms per image, while InceptionV3 demonstrated even greater efficiency, processing images at an average rate of 11 ms per image.

In this example, the viability of the present OCT probe in enhancing RCC diagnosis by improving the efficiency of PRB needle guidance has been successfully demonstrated. We applied five human kidneys and five carcinoma samples to test our system. The current outcomes yield excellent results in tissue prediction. In other non-limiting embodiments, the OCT probe can be made smaller, more portable, and user-friendly to facilitate clinical translation. By combining the deep learning approach with the forward-viewing OCT probe, a precise imaging guidance tool can be used for PRB procedures. The present probe and system have seamless compatibility with established clinical PRB guiding methods, including ultrasound, fluoroscopy, and CT. Moreover, it complements macroscopic techniques by providing high-resolution images in front of the PRB needle in real time.

### EXAMPLE 2

### Deep Learning-Aided OCT Probe for Sarcoma Biopsy Guidance

Sarcoma refers to a type of cancerous tumor which grows in soft tissue (e.g., muscle, nerve, fat, et al.) or bones. It is estimated that there will be ˜13,400 new sarcoma cases (7,400 in males and 6,000 in females) per year in the U.S. About 55% of all sarcoma cases occur in extremity, and other 45% happen in other body parts such as head, chest, abdomen, or pelvis. Biopsy is required in the diagnosis and treatment decision of sarcoma. Percutaneous core needle biopsy is an effective method for the assessment to evaluate the histologic type and grade. Medical imaging techniques are used for biopsy needle guidance. X-ray computed tomography (CT), magnetic resonance imaging (MRI) and positron emission tomography (PET) have been used as important diagnostic modalities. However, those conventional imaging modalities are limited by their low spatial resolution. It is difficult to locate the small sarcoma tumor in practical conditions. Therefore, a new imaging method is needed to improve the efficiency of sarcoma biopsy needle guidance.

### Methods

As explained above OCT is an imaging technique that has been widely applied in different medical areas. It provides micrometer level resolution and several millimeters imaging depth. Furthermore, polarization-sensitive OCT (PS-OCT) can provide more tissue contrast based on the polarization information. In this example, we built an endoscopic PS-OCT system to improve the needle guidance in sarcoma biopsy. We integrated a GRIN lens into the PS-OCT system to provide the front view of the biopsy needle and identify the tissue type in front of the needle tip. In the experiment, we used over five human extremity sarcoma samples from different body parts. PS-OCT imaging results on sarcoma tissue and normal tissue including fat and muscle in intensity mode and polarization modes have been obtained using our system. In addition, we used deep learning methods to automatically recognize the tissue type. CNN models were applied in normal tissue and sarcoma tissue classification.

### Results

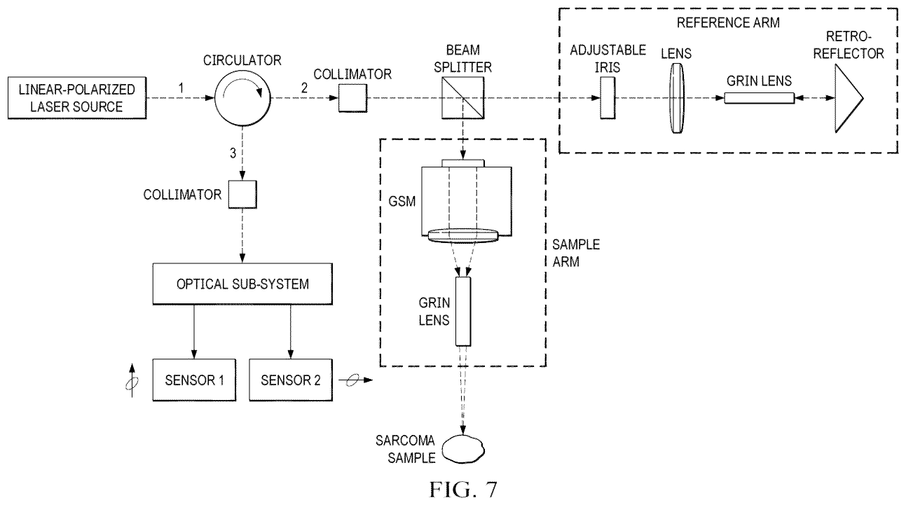

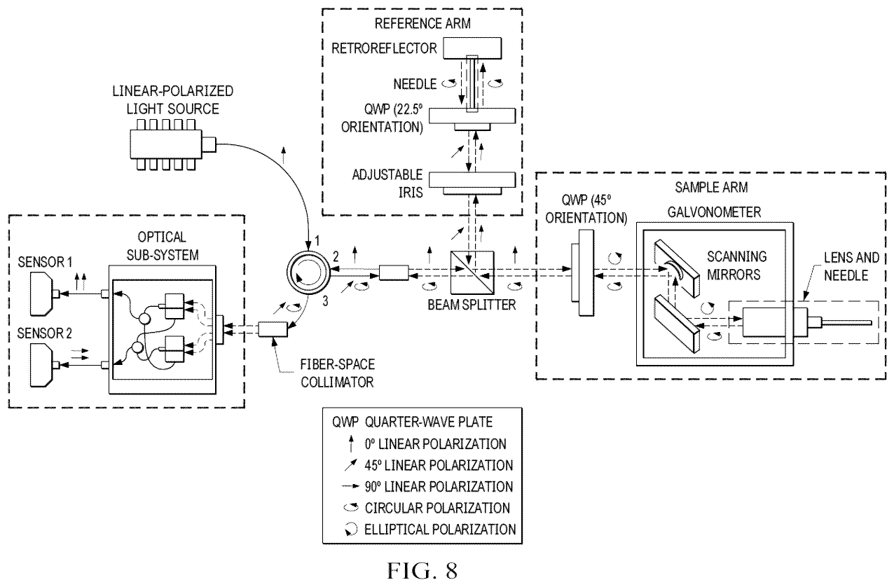

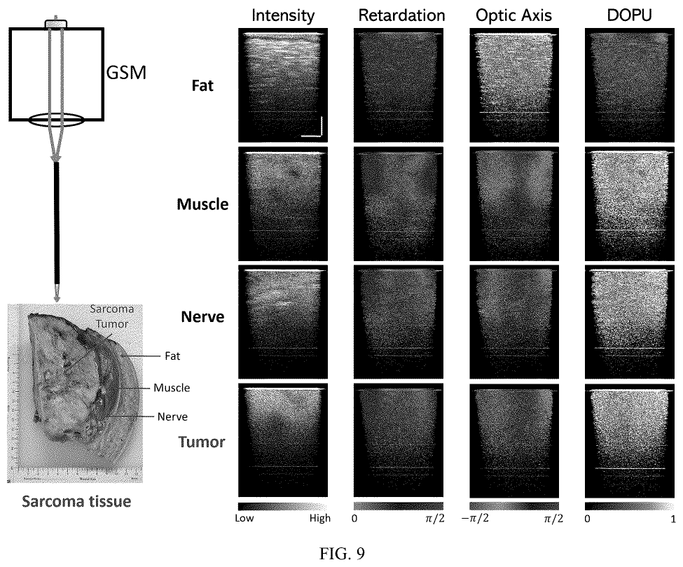

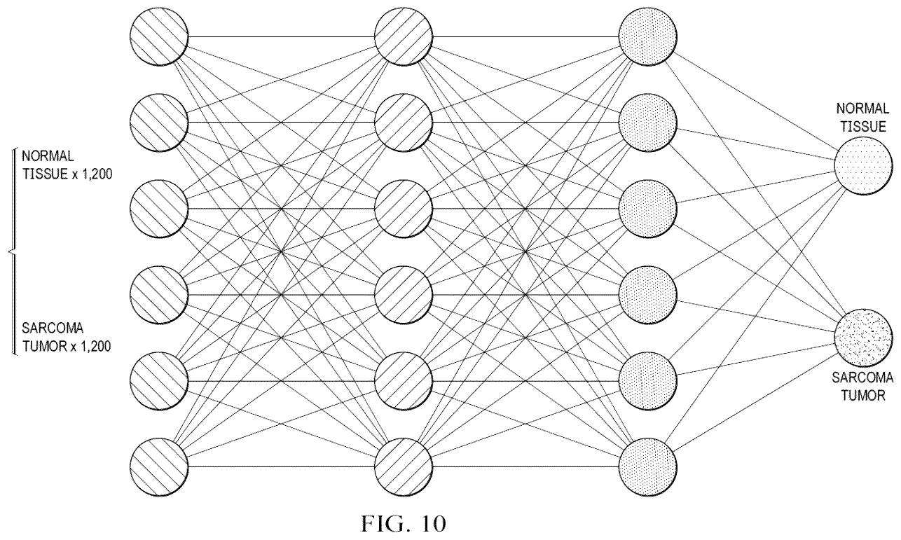

Schematic representations of the PS-OCT probe are shown in FIGS. 7-8. Imaging results of sarcoma and the surrounding normal tissues including fat, muscle, and nerve are shown in FIG. 9. In all four OCT imaging modes, sarcoma tumor and normal tissues showed different patterns on the imaging results. To automate the sarcoma recognition procedure, we used a CNN method to classify normal tissue and sarcoma tumor (FIG. 10). By using InceptionV3 architecture using the imaging results in intensity and retardation modes, validation and test accuracies both reached 100%.

In this example, we established a PS-OCT probe to help sarcoma tumor recognition. Sarcoma tumor and normal tissues showed different features in the OCT imaging results. By using the CNN method, we successfully distinguished sarcoma tumor from normal tissues.

### EXAMPLE 3

### October Probe for Liver Tumor Biopsy Guidance

Liver tumors grow on or inside the liver. It is a life-threatening disease, and the incidence rate is still growing. Around 41,000 people are estimated to get liver cancer each year and about 29,000 of them will die from the cancer. Liver biopsy is commonly used to assess the liver disease and monitor the treatment. In a standard liver biopsy, a needle is inserted inside the liver to take out a tissue sample for histological analysis. Imaging methods are required to guide the biopsy needle during insertion. Ultrasound, CT, and MRI have been utilized in liver biopsy guidance. However, due to the limited spatial resolution, it is still challenging to accurately locate the tumor position or recognize small tumors. The presently described methods using PS-OCT will aid in recognizing the tissue in front of the biopsy needle during the biopsy procedure, thereby enabling the user to avoid puncturing important normal tissues such as blood vessels.

### Methods

In this example, we used OCT to recognize the tissue in front of the biopsy needle during the biopsy procedure. We established an endoscopic PS-OCT system by integrating a GRIN lens to provide the forward view of endoscope. Five human liver tumor samples were utilized in the experiments. Normal liver tissue and tumor tissue were imaged. The PS-OCT probe provides four different imaging modes: intensity and three PS modes including retardation, optic axis and DOPU. In addition, we used the attenuation coefficient method to distinguish normal tissue and tumor tissue based on the OCT imaging results.

The schematic of the PS-OCT probe that was employed is shown in FIGS. 7-8. Two GRIN lenses were integrated in the sample arm and reference arm, respectively. The one in the sample arm is used to achieve the endoscopic imaging, and the one in the reference arm helps the dispersion compensation. This system can image the inside tissue structure by inserting the GRIN lens into the tissue.

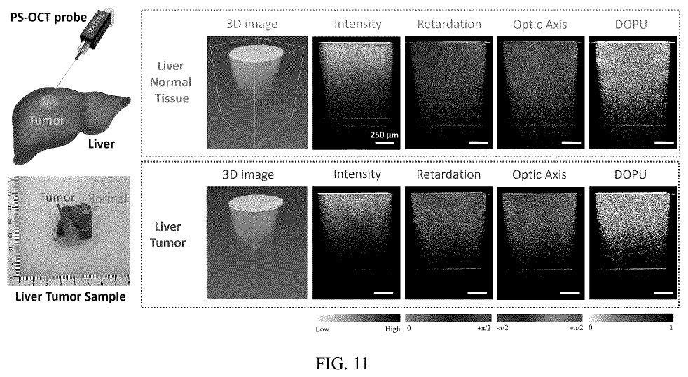

From the results shown in FIG. 11, normal liver tissue and cancerous liver tissue can be distinguished based on the imaging results. Overall, the imaging depth of the liver tumor is less than the normal tissue, because the density of tumor is higher than normal tissue. For the three PS imaging modes: retardation, optic axis, and DOPU, tumorous tissue shows different patterns when compared to normal tissues so they can further help tumor recognition in liver tumor biopsy.

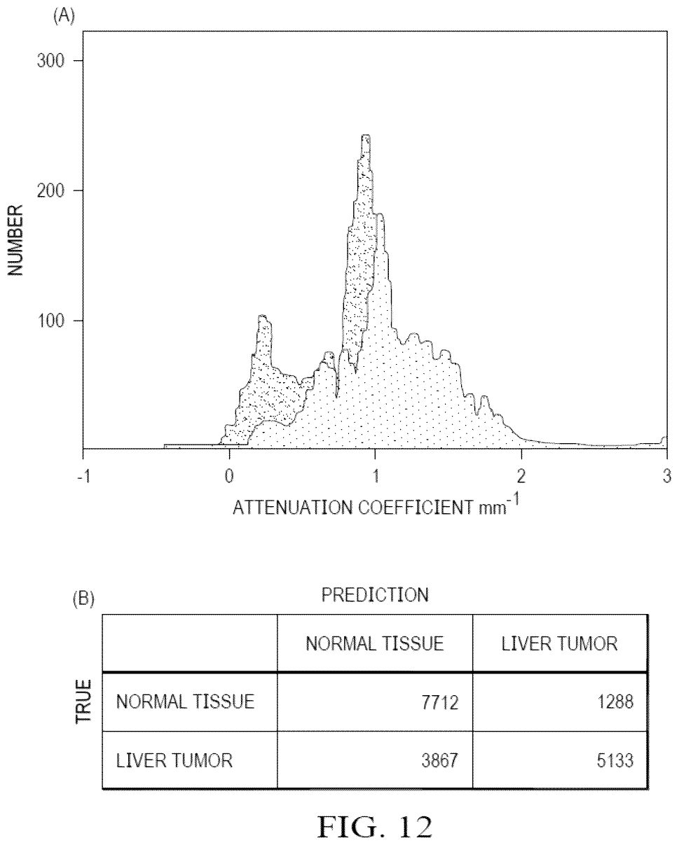

In this example, we first used attenuation coefficient method to classify liver tumor and normal tissue. Due to the different densities of tumor and normal tissue, their attenuation coefficients are different and potential to be used for tissue classification. We obtained OCT images from 5 liver tumor samples. In each sample, we acquired 1,800 liver tumor images and 1,800 normal tissue images and utilized them to calculate the attenuation coefficient. The attenuation coefficient distributions are shown in FIG. 12(A). A cross-validation method was used and the confusion matrix is shown in FIG. 12(B). The prediction accuracy reached 71.36% according to the results. In this example, we established a PS-OCT probe, and imaged human liver samples with tumor. Normal liver tissue and tumor showed different imaging results in the OCT images in different modes. We used attenuation coefficients to distinguish normal tissue and tumor, and they showed different distributions based on the results.

In summary, the present disclosure is directed, in at least one embodiment, to a method of sampling a target tissue in an animal body, comprising: obtaining an endoscope; obtaining a needle having a tip; inserting the endoscope into the needle to obtain an endoscope-needle system; inserting the endoscope-needle system into the animal body, guiding the endoscope-needle system toward the target tissue using a Doppler-based OCT system coupled with ML-CAD, wherein the Doppler-based OCT system enables the identification of blood vessels in advance of the needle tip of the endoscope-needle system whereby damage to the blood vessels is substantially avoided as the needle tip of the endoscope-needle system is guided into the target tissue; after the needle tip is guided into the target tissue, withdrawing the endoscope from the animal body while leaving the needle in situ in the animal body wherein the needle tip remains embedded in the target tissue; inserting a sampling tool into the needle; and removing a tissue sample from the target tissue. The target tissue may be a tumor selected from the group consisting of the breast, kidney, liver, bone, bone marrow, colon, small intestine, stomach, esophagus, spleen, lung, pancreas, rectum, adrenal gland, thyroid, parathyroid, pituitary, heart, muscle, peritoneum, endometrium, eye, bladder, penis, prostate, testicle, uterus, ovary, cervix, vulva, fallopian tube, skin, urethra, spine, brain, head, neck, throat, tongue, larynx, pharynx, lymph glands, thymus, nerves, fatty tissues, and central nervous system. The target tissue may be a tumor, and the OCT system may further enable the differentiation of a normal tissue from a tumorous tissue. The OCT system may be based on OCT images and CNNs. The CNNs may comprise a first CNN associated with the identification and a second CNN associated with the distance, wherein the first CNN comprises a classification model, and wherein the second CNN comprises a regression model. The needle may be selected from a Veress needle, a Tuohy needle, and a trocar. The method may further comprise performing the procedure independent of (i.e., without or absent) using a LOR technique.

Further to the above, although illustrative implementations of one or more embodiments have been provided herein, the disclosed systems and/or methods may be implemented using any number of techniques, whether or not they are currently known or in existence. The disclosed systems and methods might be embodied in many other specific forms without departing from the spirit or scope of the present disclosure. The disclosure should in no way be limited or restricted to the illustrative implementations, drawings, and techniques illustrated herein, including the exemplary designs and implementations illustrated and described herein, but may be modified within the scope of the appended non-limiting claims along with their full scope of equivalents. In addition, techniques, systems, subsystems, and methods described and illustrated in the various embodiments as discrete or separate may be combined or integrated with other systems, components, techniques, or methods without departing from the scope of the present disclosure. Other items shown or discussed as coupled may be directly coupled or may be indirectly coupled or communicating through some interface, device, or intermediate component whether electrically, mechanically, or otherwise. Other examples of changes, substitutions, and alterations are ascertainable by one skilled in the art and may be made without departing from the spirit and scope disclosed herein.

What is claimed is:

1. A method of sampling a target tissue in an animal body, comprising:
obtaining an endoscope;
obtaining a needle having a tip;
inserting the endoscope into the needle to obtain an endoscope-needle system;
inserting the endoscope-needle system into the animal body, guiding the endoscope-needle system along a path toward the target tissue using a Doppler-based optical coherence tomography (OCT) system coupled with machine-learning-based computer-aided diagnosis (ML-CAD), wherein the Doppler-based OCT system coupled with ML-CAD enables the identification of blood vessels along the path in advance of the needle tip of the endoscope-needle system thereby substantially avoiding damage to the blood vessels along the path as the needle tip of the endoscope-needle system is guided into the target tissue;
after the needle tip is guided into the target tissue, withdrawing the endoscope from the animal body while leaving the needle in situ in the animal body, wherein the needle tip remains embedded in the target tissue;
inserting a sampling tool into the needle; and
removing a tissue sample from the target tissue.

2. The method of claim 1, wherein the target tissue is a tumor selected from the group consisting of the breast, kidney, liver, bone, bone marrow, colon, small intestine, stomach, esophagus, spleen, lung, pancreas, rectum, adrenal gland, thyroid, parathyroid, pituitary, heart, muscle, peritoneum, endometrium, eye, bladder, penis, prostate, testicle, uterus, ovary, cervix, vulva, fallopian tube, skin, urethra, spine, brain, head, neck, throat, tongue, larynx, pharynx, lymph glands, thymus, nerves, fatty tissues, and central nervous system.

3. The method of claim 1, wherein the target tissue is a tumor, and the OCT system further enables the differentiation of a normal tissue from a tumorous tissue.

4. The method of claim 1, wherein the OCT system is based on OCT images and convolutional neural networks (CNNs).

5. The method of claim 4, wherein the CNNs comprise a first CNN associated with the identification and a second CNN associated with the distance, wherein the first CNN comprises a classification model, and wherein the second CNN comprises a regression model.

6. The method of claim 1, wherein the OCT system is a polarization-sensitive OCT.

7. The method of claim 1, wherein the needle is selected from a Veress needle, a Tuohy needle, and a trocar.

8. A method of sampling a target tissue in an animal body, comprising:
obtaining an endoscope;
obtaining a needle having a tip;
inserting the endoscope into the needle to obtain an endoscope-needle system;
inserting the endoscope-needle system into the animal body,
guiding the endoscope-needle system along a path toward the target tissue using a Doppler-based optical coherence tomography (OCT) system coupled with machine-learning-based computer-aided diagnosis (ML-CAD), wherein the Doppler-based OCT system coupled with ML-CAD enables the identification of blood vessels along the path in advance of the needle tip of the endoscope-needle system thereby substantially avoiding damage to the blood vessels along the path as the needle tip of the endoscope-needle system is guided into the target tissue, and wherein a loss of resistance (LOR) technique is not used as the needle tip of the endoscope-needle system is guided into the target tissue;
after the needle tip is guided into the target tissue, withdrawing the endoscope from the animal body while leaving the needle in situ in the animal body, wherein the needle tip remains embedded in the target tissue;
inserting a sampling tool into the needle; and
removing a tissue sample from the target tissue.

9. The method of claim 8, wherein the target tissue is a tumor selected from the group consisting of the breast, kidney, liver, bone, bone marrow, colon, small intestine, stomach, esophagus, spleen, lung, pancreas, rectum, adrenal gland, thyroid, parathyroid, pituitary, heart, muscle, peritoneum, endometrium, eye, bladder, penis, prostate, testicle, uterus, ovary, cervix, vulva, fallopian tube, skin, urethra, spine, brain, head, neck, throat, tongue, larynx, pharynx, lymph glands, thymus, nerves, fatty tissues, and central nervous system.

10. The method of claim 8, wherein the target tissue is a tumor, and the OCT system further enables the differentiation of a normal tissue from a tumorous tissue.

11. The method of claim 8, wherein the OCT system is based on OCT images and convolutional neural networks (CNNs).

12. The method of claim 11, wherein the CNNs comprise a first CNN associated with the identification and a second CNN associated with the distance, wherein the first CNN comprises a classification model, and wherein the second CNN comprises a regression model.

13. The method of claim 8, wherein the OCT system is a polarization-sensitive OCT.

14. The method of claim 8, wherein the needle is selected from a Veress needle, a Tuohy needle, and a trocar.
